(19) United States
(12) Patent Application Publication
(10) Pub. No.: US 2025/0290114 A1
(43) Pub. Date: Sep. 18, 2025

(54) ENUMERATION METHOD

(71) Applicant: Anabio Technologies Limited, Dublin (IE)

(72) Inventor: Sinead Bleiel, Dublin (IE)

(21) Appl. No.: 19/005,968

(22) Filed: Dec. 30, 2024

(30) Foreign Application Priority Data
Mar. 12, 2024 (GB) 2403584.2

(51) Int. Cl. C12Q 1/06; C12M 1/22

(52) U.S. Cl. CPC C12Q 1/06; C12M 23/10; G01N 2333/908

(57) ABSTRACT

A method of assaying a heat treated sample containing probiotic bacteria to enumerate viable culturable probiotic bacteria in the heat treated sample, comprises: providing a suspension comprising the heat treated sample and a nutrient broth; optionally, incubating the suspension; adding an aliquot of the incubated suspension and agar to a culture plate by a pour plate or spread plate method to provide a culture medium; incubating the culture medium for a period of time to allow probiotic bacterial colonies to grow in and/or on the culture medium; and enumerating the probiotic bacterial colonies, wherein the culture medium comprises added catalase and a pyruvate salt.

### CROSS-REFERENCE TO RELATED APPLICATIONS

This application claims benefit of priority to GB 2403584.2, filed on Mar. 12, 2024. The entire teachings of the above application is incorporated herein by reference.

### FIELD OF THE INVENTION

The present invention relates to an enumeration method. In particular, the method relates to a method of enumerating viable culturable bacteria in a heat treated sample, especially a heat treated sample containing encapsulated bacteria.

### BACKGROUND TO THE INVENTION

Enumeration of viable culturable probiotic bacteria in a heat-treated product is an important step in a product quality control process. A problem with the standard plating methods is that the probiotic bacteria are a little perturbed after heat treatment, resulting in very high variability in the numbers counted. It seems that many of the colonies in the UHT product are viable but non-culturable, which results in low recovery rates during the enumeration process. Using more advanced methods such as FACS or qPCR obviates this problem, but these processes are not recognised by regulatory authorities in many countries, which require the use of microbiological plating methods for the generation of regulatory data. This causes problems for commercialisation of such products in many countries.

It is an object of the invention to overcome at least one of the above-referenced problems. It is a particular object of the invention to provide a plate assay (e.g. pour plate or streak plate) for enumeration of viable and culturable probiotic bacteria in heat treated food products that does not exhibit the high variability in bacterial counts exhibited with standard microbiological plating methods.

### SUMMARY OF THE INVENTION

The objective is met by the provision of a modified plate enumeration method that allows for higher recovery of viable culturable cell populations. The method involves conditioning the bacterial cells in the presence of at least one neutralising agent, preferably catalase and a pyruvate salt. The neutralising agents have been found to help resuscitate the bacterial cells that have been damaged during heat treatment by supporting cell growth allowing for higher recovery of viable culturable cell populations. Data obtained indicates that the bacterial counts achieved with the method of the invention are a significant improvement compared with standard plating methods and, in some embodiment, are equivalent to more sophisticated FACS and qPCR methods of enumerating viable and culturable bacteria (e.g. exhibits bioequivalence with FACS and qPCR methods). The method is especially applicable for use with samples containing encapsulated bacteria (e.g. microparticles containing bacteria encapsulated in a polymer matrix), in which case the method comprises a step of digesting (or otherwise treating the polymer) to release the bacteria in a first incubation step, and then culturing the sample with agar on a culture plate in a second incubation step. Generally, the bacteria are conditioned with the neutralising agents during both the first and second incubation steps.

In a first aspect, the invention provides a method of assaying a heat treated sample containing bacteria to enumerate viable culturable bacteria in the heat treated sample, comprising:
providing a suspension comprising the heat treated sample and optionally a diluent (e.g. a nutrient broth) and
adding an aliquot of the suspension and agar to a culture plate by a pour plate or spread plate method to provide a culture medium;
incubating the culture medium to allow bacterial colonies to grow in the culture medium; and
enumerating the bacterial colonies,
   wherein the culture medium typically comprises added catalase and a pyruvate salt.

In any embodiment, the bacteria are probiotic bacteria.

In any embodiment, the bacteria are provided as microparticles comprising the bacteria encapsulated in a matrix (typically a protein matrix), in which the suspension comprising the heat treated sample comprises a protease enzyme and optionally a diluent (e.g. a nutrient broth), and in which the method includes a step of incubating the suspension in a first incubation step to release bacteria from the microparticles.

In any embodiment, the diluent has a pH of 5.4 to 7.2.

In any embodiment, the diluent is selected from a nutrient broth and a buffered aqueous solution (e.g. phosphate buffered saline).

In any embodiment, the concentration of added catalase in the culture medium is 100-250 U per millilitre culture medium.

In any embodiment, the concentration of the pyruvate salt in the culture medium is 10-30 mM, 15-25 mM, and ideally about 20 mM.

In any embodiment, the catalase and/or pyruvate salt is added to the suspension.

In any embodiment, the catalase and/or pyruvate salt is added to the agar.

In any embodiment, the catalase and pyruvate salt is added to the suspension and to the agar. In this embodiment, the bacterial cells are incubated with catalase and a pyruvate salt for an extended period, e.g. during the (optional) digestion step and during the culturing step.

In any embodiment, the suspension is homogenised before and/or after the first incubation step. The function of pre and/or post homogenisation acts as a shear force to break down the protein matrix of the microparticle, to release the cells. The pre homogenisation aids the mechanical erosion of the micro particle matrix and increase the surface area exposure of the micro particle to enhance the enzyme site activity for protein digestion. The post homogenisation facilitates the full disintegration of soluble aggregates that may remain after initial treatment and ensure that the probiotics are free and liberated from protein microparticle matrix. This phase acts to removes the last large, solubilised agglomerates (produced as a result of the digestion), and break up Lactobacillus chains to enable cell growth of individual colonies on agar plate thereafter. The or each homogenisation step may be performed using a stomacher or a ultra-turrax high shear mixer.

In any embodiment, the protease is combined with the heat treated sample and a diluent (e.g. a nutrient broth) during the first incubation step. Typically, the first incubation step comprises a first stage prior to addition of protease (1-6 hours) and a second stage which begins when the protease is added (30-90 minutes). For example, the protease may be added after 50-90% or 50-75% of the first incubation step has elapsed. The optimal time for the second stage of the first incubation step is 20-40, preferably 30, minutes, at 30-40° C., ideally at 37° C. The protease has an optimal pH for the process between 5.9-7.1 however it can be used in a range of 5.5-7.5 and we still get >90% enzyme activity. Typically the suspension is stirred during the first incubation step, suitable at 200-300 RPM, ideally about 250 RPM. Typically, the first incubation step is performed in a first incubation vessel containing a headspace containing oxygen enriched air (e.g. 20-25% oxygen, ideally about 22% oxygen v/v). The diluent may be selected from a nutrient broth or a buffered saline (e.g. phosphate buffered saline having a phosphate concentration of 50-100 mM). Typically, the diluent has a pH of 5.4-7.2.

In any embodiment, the aliquot of the incubated suspension is prepared by serial dilution. In any embodiment, the method comprises assaying multiple aliquots of the suspension prepared by serial dilution.

In any embodiment, the culture medium comprises added L-cysteine. The L-cysteine may be present in the suspension, the agar, or both.

In any embodiment, the culture medium comprises added D-(+) Trehalose. The D-(+) Trehalose may be present in the suspension, the agar, or both.

In any embodiment, the heat treated sample is a UHT beverage.

In any embodiment, the probiotic bacteria are selected from Bifidobacteria and Lactobacillus.

In any embodiment, the added catalase is added to the agar when the agar has a temperature of 40 to 50° C., typically 40-45°, and ideally about 42-43° C.

In a preferred embodiment, the culture plate is prepared by the spread plate method, in which the method comprises the steps of pouring a first layer of agar, allowing the first agar layer to solidify, spreading the aliquot of the incubated suspension on a top of the first agar layer, and pouring a second agar layer on top of the first agar layer.

In any embodiment:
the catalase and pyruvate salt are added to the suspension and the agar;
the concentration of added catalase in the culture medium is 100-250 U per millilitre culture medium; and/or the concentration of the pyruvate salt in the culture medium is 10-30 mM.

In another aspect, there is provided a culture medium comprising agar, nutrient broth, added catalase, added pyruvate salt, and an inoculum comprising bacteria.

In any embodiment, the culture medium comprises 100-250 U catalase per millilitre of culture medium and 10-30 mM pyruvate salt.

In any embodiment, the inoculum comprises a hydrolysate comprising digested, ideally protease-digested, bacteria-containing microparticles (e.g. a hydrolysate obtained by digesting microparticles comprising bacteria encapsulated in a polymer (e.g. protein) matrix with a protease).

In any embodiment, the culture medium comprises added L-cysteine. The presence of cysteine 1) supports cell resuscitation during incubation but it also 2) helps to stabilise the catalase during both the suspensions stage and 3) immobilise catalase in the agar or help growth of cells in the agar during final incubation on spread or pour plates. Thus, while the presence of L-cysteine may not be directly involved in the enzyme's catalytic activity with hydrogen peroxide in the analytical procedure, it is helps maintain the structural integrity of catalase for regulatory processes within the cell for growth on the agar.

In another aspect, the invention provides a culture plate comprising a culture medium of the invention. The culture plate may be, for example, a Petri dish.

In any embodiment, the culture plate is prepared by a pour plate or spread plate method.

Other aspects and preferred embodiments of the invention are defined and described in the other claims set out below.

### BRIEF DESCRIPTION OF THE FIGURES

FIG. 1A illustrates the procedure for analysing a heat treated sample using a spread plate method according to the method of the invention. This method involves extracting the probiotic bacteria from the food matrix by producing a suspension with a nutrient broth and a protease enzyme.

FIG. 1B illustrates the plate that is prepared with an agar supplemented with sodium pyruvate according to the method of the invention. The catalase is spread on the surface of the solidified agar with a sterile hockey stick and following this the sample is spread on the surface of the agar with immobilised catalase.

FIG. 1C illustrates procedure for analysing a heat treated sample using a spread plate method with an additional enzyme digestion step according to the method of the invention. After the incubation period (2.5 hours), the protease is added and a further incubation step (30 min) is carried out.

FIG. 2A illustrates one embodiment of the method of the invention that employs an agar overlay. This agar overlay is added on the agar plate with the immobilised catalase and the sample which creates a protective layer for the enzyme by limiting the oxygen exposure.

FIG. 2B illustrates one embodiment of the method of the invention in which the agar plate with immobilised catalase and the sample spread on top of the agar plate with added sodium pyruvate with the addition of the agar overlay.

FIG. 2C illustrates one embodiment of the method of the invention that employs a spread plate method with the additional steps of an enzyme digestion step and an agar overlay step.

FIG. 3A illustrates one embodiment of the method of the invention that employs a spread plate method with immobilised catalase and sodium pyruvate on the agar plate.

FIG. 3B illustrates one embodiment of the method of the invention that employs a plate that is prepared with immobilised catalase, sodium pyruvate to allow direct contact of the sample and an additional agar overlay step.

FIG. 3C illustrates one embodiment of the method of the invention that employs a spread plate method with immobilised catalase and sodium pyruvate on the agar plate. This also shows the additional enzyme digestion step.

FIG. 4A illustrates one embodiment of the method of the invention that employs a pour plate method wherein the suspension containing the sample, catalase and sodium pyruvate is placed in an empty petri dish with tempered agar added thereafter and is left to solidify.

FIG. 4B illustrates one embodiment of the method of the invention that employs a pour plate method with the agar which contains the sample, catalase and sodium pyruvate distributed throughout the agar plate.

FIG. 4C illustrates one embodiment of the method of the invention that employs a pour plate method wherein the suspension containing the sample, catalase and sodium pyruvate is placed in an empty petri dish with tempered agar added thereafter and is left to solidify with an additional enzyme digestion step.

FIG. 5A illustrates one embodiment of the method of the invention that employs a pour plate method wherein the sample is placed in the empty petri dish and the tempered agar which is supplemented with catalase and sodium pyruvate.

FIG. 5B illustrates the agar plate prepared by the pour plate method showing the catalase, sodium pyruvate and the sample distributed throughout the agar plate.

FIG. 5C illustrates the procedure for analysing a heat treated sample using a pour plate method wherein the sample is placed in the empty petri dish and the tempered agar which is supplemented with catalase and sodium pyruvate with an additional enzyme digestion step.

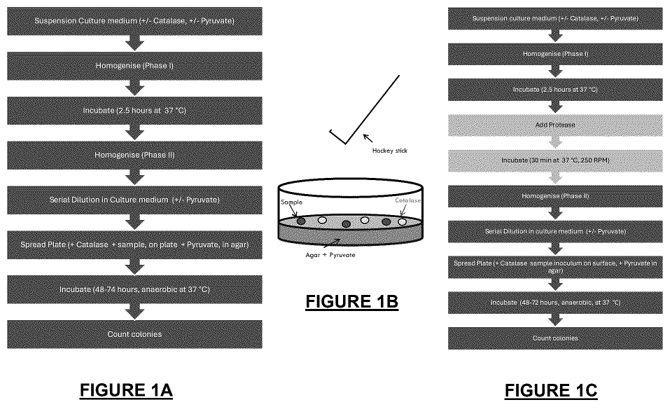

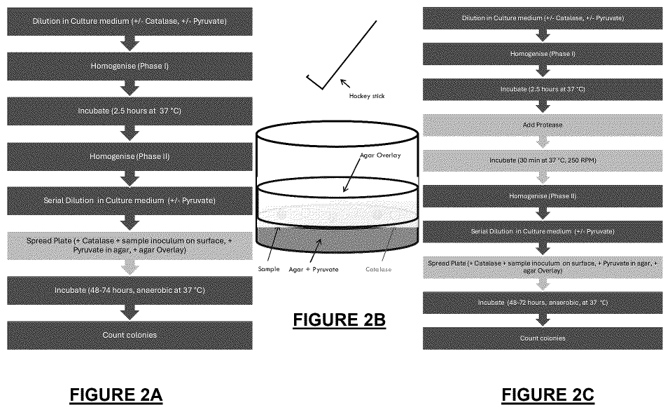

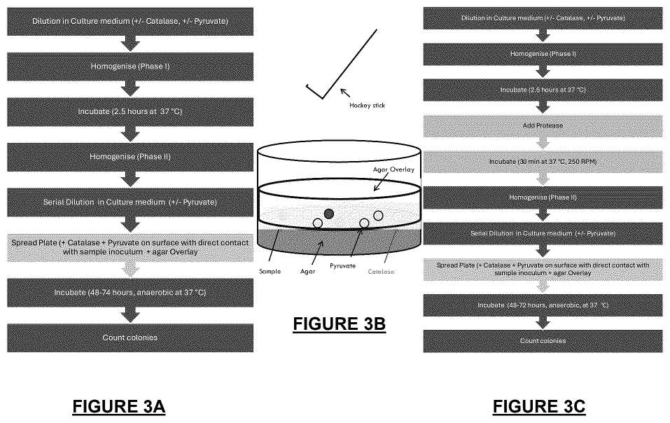

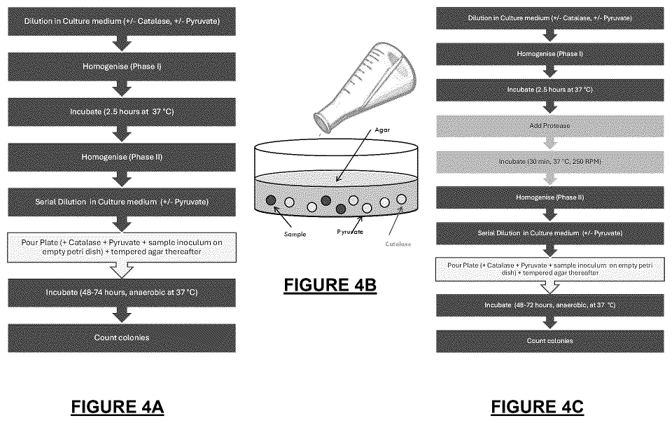

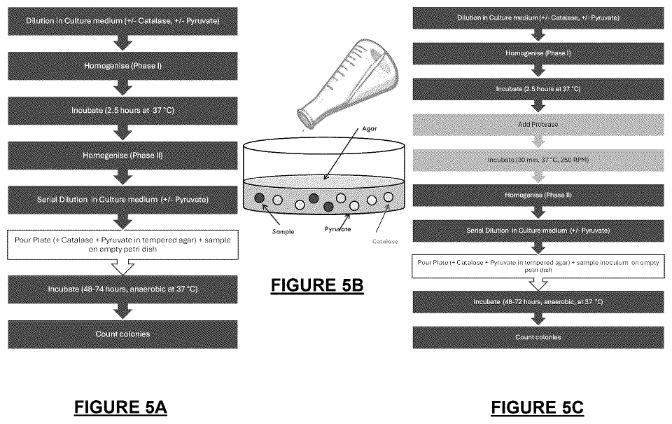

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

### Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

As used herein the term “viable” refers to microbial cells that are physiologically active and capable of conferring a benefit to the consumer.

As used herein the term “culturable” refers to microorganism capable of isolation using a culture preparation approach.

As used herein the term “catalase” refers to an enzyme which is found in nearly all living organisms exposed to oxygen. Catalase breaks down hydrogen peroxide into water and oxygen. It is commonly used in microbiology to differentiate between bacterial species based on their ability to produce catalase. This invention utilises catalase to promote the cultivation of probiotics after encapsulation and processing in a food product. There are various sources of catalase including bovine (liver) and fungi such as Aspergillus niger. The enzyme may be a wild-type or recombinant enzyme. In this invention, an effective catalase concentration is 100-250 units per millilitre of culture medium. This concentration is typically sufficient to degrade hydrogen peroxide effectively for probiotics after encapsulation and fortification into a food product. The exact amount of catalase to add will depend on the volume of agar used. For instance, if 15 mL of agar per plate is required, and catalase concentration required is 1000 units/mL, 15,000 units of catalase per plate is added. The current invention utilises 5000 IU per sample either in suspension and/or on an agar plate. Catalase is listed as an ingredient for use in certain fermented products. For example, it is approved for use in cheese manufacturing, as indicated by the U.S. FDA, demonstrating its role in the food industry, including fermented foods. Additionally, catalase from bovine liver is recognized as Generally Recognized as Safe (GRAS) for use in decomposing hydrogen peroxide. The catalase typically needs at least 90 minutes to work in the suspension; 2-3, or about 2.5, hours is the optimal time for catalase. And 35-40° C., typically about 37° C. is the optimal temperature for catalase to work and also for the probiotics to resuscitate. Room temperature can be used but there is marginal impact on resuscitation hence why 37° C. is optimal to support the cell recovery while using a temperature close to the optimal temp for catalase. Catalase can be added to suspension for a time anywhere between 30 minutes to 21 hours at, for example, 27° C.

As used herein the term “pyruvate salt” refers to a salt of pyruvate, is a key intermediate in several metabolic pathways, including glycolysis. The salt is generally a sodium salts but the use of other salts such as potassium is also possible. Pyruvate plays a pivotal role in energy production processes within the cell. It can be converted into acetyl-CoA and enter the citric acid cycle (Krebs cycle) for further energy production, or it can be used in the process of gluconeogenesis to produce glucose.

As used herein the term “protease” refers to an enzyme that is used to break down proteins by hydrolyzing peptide bonds and is used in the invention to degrade the protein components of the micro-particle matrix. Proteolytic enzymes are known to possess catalytic, non-catalytic and ancillary domains. Proteases are broadly classified as endopeptidases and exopeptidases. The protease used herein is preferably an endo-protease of the serine type. It has a broad substrate specificity and can hydrolyze most peptide bonds within a protein molecule. It is active between pH 6.5 and 8.5 and has an optimum temperature of 60° C. The enzyme is used in detergent formulations to remove protein-based stains. Protease catabolizes proteins by hydrolysis of peptide bonds. Proteases are inactivated by serine active-site inhibitors, such as phenylmethylsulfonyl fluoride (PMSF) and diisopropylfluorophosphate. The protease used herein is typically sourced from Bacillus licheniformis or Proteinase from Bacillus licheniformis Subtilisin A.

The protease is generally employed at about 0.05 to 0.3%, ideally about 0.1 to 0.2%. This can be adjusted slightly depending on product matrix. For example of the product is a high protein drink, 0.2-0.22% protease may be employed to help digest the protein in the product and also the protein microparticles (all v/v).

As used herein the term “sample” refers to a comestible product such as a food, beverage or supplement. The food product may be a confectionary product such as a gummy or gelled matrix type product (e.g. a pectin cube or snack), a nutritional syrup and beverages, a food snack, a diary product. The beverage can be, for example, a beverage comprising one or more of fruit juices, fruit drinks, fruit juice, fruit drink or fruit containing products such as mixed fruit juice/dairy drinks, mixed fruit juice/non-dairy drinks, mixed fruit juice/dairy food products, mixed fruit juice/non-dairy food products, mixed fruit drink/dairy drinks, mixed fruit drink/non-dairy drinks, mixed fruit drink/dairy food products, mixed fruit drink/non-dairy food products, mixed fruit/dairy drinks, mixed fruit/non-dairy drinks, mixed fruit/dairy food products, mixed fruit/non-dairy food products, yoghurt and yoghurt drinks. Preferably, the pH of the sample is within 2.0-7.8. Preferably, the protein content of the sample is 0.1%-12%. Preferably, the sample is fortified with encapsulated probiotic bacteria with a bacterial count of between 10⁶ and 10¹⁰ CFU/mL or CFU/g and a carrier medium (beverage or gum matrix).

As used herein the term “heat treated” as applied to the sample refers to samples that have been subjected to a heat-treatment process during preparation, to prolong the shelf life of the product (such as UHT) or as part of the manufacturing process (such as gel-type products that have to be heated and gelled). There are different heat treatments employed in the food industry for different purposes. Examples of heated treatments include but are not limited to, pasteurisation which involves heating the product below 100° C., tunnel or flash pasteurisation, and ultra-heat treatment (UHT).

As used herein the term “UHT” refers to ultra-heat treatment involves heating the product to higher temperatures for short holding times. Typical UHT treatments involve heating a product to a temperature of greater than 135° C. for 2-5 seconds.

As used herein the term “probiotic bacteria” refers to live beneficial microorganisms that provide health benefits to the host by restoring a healthy level of beneficial bacteria in the gut. Examples of commercially available probiotic bacteria include Lactobacillus rhamnosus GG, Bifidobacterium lactis (Activa), Bifidobacterium infantis 35624 (Align), Lactobacillus GG (Culturelle), Lactobacillus rhamnosus GR-1), Lactobacillus reuteri RC-14 and Lactobacillus acidophilus LA-5 (Trubiotics).

As used herein the term “plate method” refers to a technique that is employed in the field of microbiology to isolate and analyse microorganisms. There are various known techniques. The pour plate technique involves mixing the sample (usually the sample containing a nutrient broth) and the liquid agar followed by solidifying and then an incubation period. The spread plate method involves preparing a culture plate with agar and spreading the sample on the surface of the agar with the use of a sterile hockey stick. This method allows accurate enumeration and isolation of colonies for further analysis including but not limited to PCR analysis, genome sequencing, 16S blast. The pour plate method as used herein is useful for enumerating viable and culturable probiotic colonies and can support quantification of H₂O₂ decomposition via quantification of water generated. The spread plate method is versatile, and useful for counting viable and culturable probiotic cells for further sequencing requirements. As used herein, both spread and pour plate methods enable the decomposition of H₂O₂ in the agar environment to support optimal cultivation of the probiotic cells in the agar matrix. Spread plate permits for easier identification and isolation of the colonies for further testing. Conversely, pour plate is more suitable for higher throughput settings for example in large scale quantities as used in an industrial setting.

As used herein the term “culture medium” refers to the contents of a culture plate, e.g. the sample, nutrient broth, agar and one or more neutralising agents such as catalase and/or a pyruvate salt.

As used herein the term “neutralising agent” refers to a compound that minimises oxidative stress on microbial cells by neutralising reactive oxygen species generated during growth. Examples include catalase and a pyruvate salt. Ideally, the method comprises incubating the sample with both catalase and pyruvate salt. Preferably, the method comprises incubating the bacteria with the neutralising agent(s) during the second incubation step, and ideally also during the first incubation step.

As used herein, the term “added” as applied to the neutralising agent(s) (e.g. catalase and a pyruvate salt) means that the neutralising agent is exogenous, e.g it is added into the method as opposed to be endogenous (e.g. from the bacterium).

As used herein the term “agar” refers to MRS agar, and other selective agars and broths used for lactic acid bacteria cultivation and enumeration. “MRS agar” or de Man, Rogosa, and Sharpe agar refers to a medium commonly used for cultivating lactic acid bacteria (LAB). The formulation of MRS agar is specifically designed to promote the growth of LAB by providing a rich nutrient environment, typically including peptone, beef extract, yeast extract, glucose, and other components conducive to LAB growth. MRS agar does not inherently contain hydrogen peroxide.

As used herein the term “microparticles” refers to a particle comprising bacteria (ideally probiotic bacteria) encapsuled in a matrix which is usually a protein such as a plant or milk protein. The microparticles can, for example, be formed by cold gelation as described in WO2010119041A2, WO2016096929A1 and WO 2022-023390A1 or using a fluidised bed as described in WO2023144354A1. When the sample being assayed comprises microparticles, the method comprises a first incubation step in which the sample is incubated with a protease for a time sufficient to allow the protease digest the polymer (e.g. protein) matrix and release the bacterial contents. This first incubation step is typically carried out in the presence of a neutralising agent(s). Typically, the first incubations step comprises a first stage in which the sample and neutralising agent(s) are incubated, and a second stage that begins when the protease is added. The first stage may be 1-6 hours, 1-5 hours, or 2-5 hours. The second stage may be 15-90 minutes, 15-60 minutes, 30-60 minutes. The first incubation step is generally performed at 30-40° C.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### Preparation of Catalase Immobilised Plates for Extraction and Enumeration

Preparation of Catalase Solution with Sterile Water:

### Materials/Reagents:

Catalase from bovine liver (Units 2000-5000/mg)
Sterile water

### Consumables:

Vacuum Filtration System with 0.22 μm filter
Sterile sample container
Spatula
25 ml serological pipette

### Protocol

Clean a spatula with IPA (alcohol)
Weigh 0.25 g of catalase inside a sterile sample container.
Transfer the container to a laminar flow hood.
Add 99.75 mL of sterile water using a 25 ml serological pipette.
Invert the solution 10 times to mix thoroughly and vortex at low speed for 10-20 seconds.
Incubate the solution for at least 30 minutes at room temperature.
After incubation, vortex the solution 5 seconds at low speed.
Filtrate through 0.22 μm.

### Preparation of Catalase Solution Potassium Phosphate (pH 7.5);

### Materials/Reagents:

Catalase from bovine liver (Units 2000-5000/mg)
Potassium phosphate dibasic
Potassium Phosphate monobasic
Sterile water
IPA

### Consumables:

Vacuum Filtration System with 0.22 μm filter
Sterile sample container
Spatula
25 mL serological pipette

### Protocol:

Prepare the potassium phosphate buffer (pH 7.5) by adding 800 mL of sterile water into a Duran bottle. Add 12.813 g of Potassium phosphate dibasic and 3.598 g of Potassium phosphate monobasic. Add sterile water to bring the solution to 1 L. Mix with a stir bar for 20 minutes at room temperature.
Clean a spatula with IPA and weigh 0.25 g of catalase inside a sterile sample container.
Transfer the container to a laminar flow hood and add 99.75 g m of potassium phosphate buffer using a 25 mL serological pipette.
Invert the solution 10 times to mix thoroughly and vortex at low speed for 10-20 seconds.
Agitate the solution for at least 180 minutes at room temperature at 200 RPM
After full hydration, transfer solution to the vacuum filtration system.
Filtrate the solution through 0.22 μm.

### Preparation of 2 M Sodium Pyruvate:

### Materials/Reagents:

Sodium Pyruvate
Sterile water

### Consumables:

Vacuum Filtration System with 0.22 μm filter
Sterile sample container
Spatula
25 mL serological pipette

### Protocol:

Clean a spatula with IPA.
Weigh sodium pyruvate inside a sterile sample container.
Transfer the container to a laminar flow hood and add sterile water to create a 2 M Solution.
Invert the solution 10 times to mix thoroughly and vortex at low speed for 10 to 20 seconds.
Incubate the solution for at least 30 minutes at room temperature.
After incubation, vortex the solution for 5 seconds at low speed
Filter through 0.22 μm unit filter.
The solution is stored at 4° C. when not in use and expires on the same day of preparation.
   Preparation of L-Cysteine HCl Stock Solution (5% w/v):

### Materials/Reagents:

L-Cysteine Hydrochloride Monohydrate
Sterile water.

### Consumables:

Vacuum Filtration System with 0.22 μm filter.
Sterile sample container.
Spatula.
25 ml serological pipette.

### Protocol:

Dissolve 5 g L-Cysteine-HCl in 95 mL RO water.
Filter through 0.22 μm
This represents 5% v/v L-Cysteine HCl stock solution and can be stored at 4 to 8° C. for a maximum of 7 days.

### Catalase Immobilised Plates

The preparation of catalase immobilised pour plate and spread plate are two distinct methods, each with its own protocol for preparing and analysing encapsulated samples from fortified products. Here are the key differences between these two methods.

The pour plate method (in the context of finished product probiotic analysis) is specific for identifying probiotic colonies and can support quantification of H₂O₂ decomposition via quantification of water generated, while the spread plate method is versatile, used for isolating and counting viable probiotic cells for further sequencing requirements.

Both spread and pour plate enable the decomposition of H₂O₂ in the agar environment to support optimal cultivation of the probiotic cells in the agar matrix. Each method serves the same invention needs; however, spread plate permits for easier identification and isolation of the colonies for further testing. Conversely, pour plate is more suitable for higher throughput settings.

### Materials Required for Either Pour or Spread Plates of Immobilised Catalase:

Catalase enzyme: Pure catalase enzyme, preferably in a powdered form or concentrated solution.
Sodium Pyruvate: Pyruvi acid sodium salt (CH₃COCOONa).
L-Cysteine L-Cysteine hydrochloride monohydrate: L form (HSCH₂CH(NH₂)COOH·HCl·H₂O).
Agar (MRS/Rogosa/TOS): Agar powder for preparing the gel for Lactobacillus cultivation.
Buffer solution: Phosphate buffer or any suitable buffer to maintain an appropriate pH for catalase activity, typically around pH 7.0/Potassium Phosphate.
Petri dishes: For pouring the agar gel.

### Catalase Immobilised Pour Plate Method

Catalase immobilised pour plate method is designed to resuscitate probiotics after extraction from a beverage or food product. Catalase is typically added to an agar to differentiate between catalase-positive (e.g., Staphylococcus spp.) and catalase-negative bacteria (e.g., Streptococcus spp.). However, the purpose of this invention is significantly different. The addition of immobilised catalase to a pour plate provides for the most suitable environment to allow for the resuscitation of probiotics on an agar plate. Lactobacillus are catalase negative hence this method does not promote the production of catalase by the probiotics; the addition of catalase supports the decomposition of H₂O₂ in the agar to enable the better growth and resuscitation of cells on the agar plate. Cells are englobed in the agar matrix and colony counting is performed (acceptable counts between 30-300 colonies).

### Steps

Prepare a buffer solution to maintain the pH that is optimal for catalase activity. The pH should be around 7.0, which is typically ideal for catalase. Optimal Buffer for catalase is Potassium Phosphate 100 mM prepared in water.
Prepare a catalase Stock Solution. If catalase enzyme is to be added to the agar, it should be in a purified form, diluted appropriately in water or appropriate buffer to maintain its activity.
Preparation of sodium pyruvate stock solution: Prepare sodium pyruvate in water and filter sterilise through 0.22 μm. Concentration will be dependent on the agar bottle volume.
Preparation of L-Cysteine stock solution: Prepare 5% w/w L-Cysteine in water and filter sterilise through 0.22 μm.
Prepare an agar medium and cool it to 42.5° C. after autoclaving at 121° C. for 15 minutes. Agar that is too hot can denature proteins, including enzymes like catalase, rendering them inactive.
Once the agar has cooled to a safe temperature that maintains enzyme activity, gently mix the catalase solution into the liquid agar. It is crucial to carry out this step quickly and efficiently to prevent pre-mature solidification of the agar and ensure uniform distribution of the enzyme in the agar solution.
Immediately pour the catalase-containing agar into Petri dishes with the extracted sample suspension. The agar must be poured before it begins to solidify to ensure an even spread and effective incorporation of the enzyme throughout the medium for efficient immobilisation of the catalase and sample.
Proceed with incubation as per your experimental design for lactic acid bacteria, typically 37° C. for 48-72 hours.
Colony appearance will not be affected by the presence of catalase in the agar; however, if the activity of catalase will decompose H₂O₂ to H₂O and O₂, additional water will be generated in the plates after incubation.

### Test to Verify Catalase Enzyme Activity:

Once the agar solidifies, hydrogen peroxide can be added to the surface to test for catalase activity. This is qualitatively and quantitatively defined by the time, height extension and foam ability of the plate.
The production of bubbles (oxygen gas) indicates presence of catalase enzyme at the specified time and temperature correlated with the enzyme activity.

Enzyme activity can be measured via colorimetric assay methods based on the measurement of the hydrogen peroxide substrate remaining after the action of catalase. Most colorometric kits operated on basic principles as follows: First, the catalase converts hydrogen peroxide to water and oxygen (catalytic pathway) and then this enzymatic reaction is stopped with sodium azide. An aliquot of the reaction mix is then assayed for the amount of hydrogen peroxide remaining by a colorimetric method. The colorimetric method uses a substituted phenol (3,5-dichloro-2-hydroxybenzenesulfonic acid), which couples oxidatively to 4-aminoantipyrine in the presence of hydrogen peroxide and horseradish peroxidase (HRP) to give a red quinoneimine dye (N-(4-antipyryl)-3-chloro-5-sulfonatep-benzoquinone-monoimine) that absorbs at 520 nm. Here are some examples of fluorescent assays used for catalase quantification:

### Amplex Red Hydrogen Peroxide/Peroxidase Assay:

Amplex Red reagent, which reacts with hydrogen peroxide in presence of horseradish peroxidase (HRP) to produce a fluorescent product, resorufin i.e. decrease in hydrogen peroxide concentration due to catalase activity can be indirectly measured by monitoring the reduction in fluorescence

### DCFDA Assay (2′,7′-Dichlorofluorescein Diacetate):

DCFDA is oxidized by hydrogen peroxide to form the highly fluorescent compound DCF (2′,7′-dichlorofluorescein) i.e. decrease in fluorescence intensity indicates the breakdown of hydrogen peroxide by catalase

### Hydrogen Peroxide-Sensitive Fluorescent Probes

Peroxy Orange 1 (PO1) and Peroxy Green 1 (PG1) fluoresce upon reaction with hydrogen peroxide i.e. reduction in fluorescence due to catalase activity can be used to quantify enzyme activity

### Amplex Red Hydrogen Peroxide/Peroxidase Assay:

Prepare Reaction Mixture: Combine the sample with catalase with hydrogen peroxide in a suitable buffer (not sure what buffer we need?)
Add Amplex Red Reagent: Add the Amplex Red reagent and horseradish peroxidase (HRP) to the reaction mixture.
Incubate: Allow the reaction to incubate at room temperature for 30 min
Measure Fluorescence: Measure fluorescence @ excitation wavelength 530 nm/emission wavelength 590 nm
Calculate Catalase Activity: Compare the fluorescence intensity to a standard curve prepared with known concentrations of hydrogen peroxide.

### Advantages of Amplex Red Hydrogen Peroxide/Peroxidase Assay

High Sensitivity: The assay is highly sensitive and can detect low levels of hydrogen peroxide
Ease of Use: The protocol is straightforward and typically involves adding the Amplex Red reagent to your sample, incubating, and measuring fluorescence.
Stable Signal: The fluorescent product, resorufin, is stable and provides consistent
Wide Dynamic Range: It can accurately measure a broad range of hydrogen peroxide concentrations.
   Catalase immobilised Spread Plate Method

This catalase immobilised spread plate technique is primarily used for the enumeration of probiotic cells extracted from heat treated products which are fortified with encapsulated probiotics and require isolation of individual colonies for further analysis of specific colonies i.e. PCR analysis, genome sequencing, 16S blast, etc.

### Procedure:

Prepare a buffer solution to maintain the pH that is optimal for catalase activity. The pH should be around 7.0, which is typically ideal for catalase. Optimal buffer for catalase is Potassium Phosphate 100 mM prepared in water.
Preparation of sodium pyruvate stock solution: Prepare sodium pyruvate in water and filter sterilise through 0.22 μm. Concentration will be dependent on the agar bottle volume.
Preparation of L-Cysteine stock solution: Prepare 5% w/w L-Cysteine in water and filter sterilise through 0.22 μm.
Preparation of catalase stock solution: Catalase is hydrated in buffer at room temperature for 180 minutes and filter sterilise through 0.22 μm. Catalase is prepared on day of use. Catalase can also be prepared in water, however; the invention promotes for the use of catalase in buffer to avoid variability in plate counts due to difference in finished product pH.
Dissolve agar powder in the buffer solution by heating it in a microwave or on a hot plate, following the concentration instructions provided by the agar manufacturer. The final concentration of agar should be about 1.5% for a firm gel. de Man, Rogosa and Sharpe (MRS), Rogosa, Nutrient and (Transgalctosylated oligosaccharide-propionate (TOS) agar is both typically used as standard growth media for cultivation of lactic acid bacteria from various food source.
Allow the agar solution to cool down to 42.5° C., a temperature that is warm enough to keep the agar liquid but cool enough not to denature the catalase enzyme.
Add sodium pyruvate at concentration of 20 mM per bottle (filter sterilised through 0.22 μm filter) from stock solution prepared in water.
Add L-Cysteine at 0.05% concentration per bottle (from filter sterilised (0.22 μm filter) stock solution prepared in water.
Gently mix the agar solution after addition of the additives (pyruvate and L-Cysteine) and pour molten agar into Petri dishes. Work quickly to avoid premature solidification of the agar.
Solidify and Store: Allow the agar to solidify at room temperature for 20 minutes.
Add catalase solution to the surface each plate (2,000-5,000 IU per plate) at each dilution and distribute with a hockey stick until evenly dispersed without touching walls of the plate.
Allow the plates the dry for 10-15 min at room temperature until the surface is dry.
Add extracted probiotic test sample to the catalase immobilised plate, spread sample with hockey stick and dry for 10 min.
If plates are not used immediately, can be stored in a refrigerator. Extreme cold can also affect enzyme activity, so it is advisable to bring the plates to room temperature before use.
After incubation, individual colonies can be counted or sub cultured for further identification or analysis.

### Food Product Sample Extraction and Enumeration of Probiotics

TABLE 1
Equipment required.

| Equipment and Material | Purpose |
|---|---|
| Stirring plate | Agitation of sample during incubation |
| Incubator | Heating of sample during incubation |
| 3 cm, ⅞^{th} inch Stir bar | Agitation of sample during incubation |
| Autoclave | Sterilisation of reagents |
| Schott Duran 100 mL | Sample container |
| Microlitre pipette | Serial dilutions (1000 series) |
| Petri dishes | Plating of sample |
| Test tubes | Serial dilutions |
| Vortex | Agitation of sample |
| Water bath x2 | Melting/tempering media |
| Anaerobic jars | Required for anaerobic incubation |
| Stomacher | Stomacher 400 |
| Stomacher Bags | Sterile Stomacher Bags; 400 mL fill capacity |
| 0.22 μm Vacuum filter | Filter Sterilization |

TABLE 2
Materials required.

| Reagent | Concentration |
|---|---|
| MRS agar (for Lb) | As per manufacturer's instructions |
| ToS Media (for Bif) | As per manufacturer's instructions |
| Alternative agar | |
| Protease from Bacillus lichenformis | Enzymatic degradation in |
| | suspension |
| MRS broth | As per manufacturer's instructions |
| L-Cysteine Hydrochloride | Preparation of 5% (v/w) solution - |
| Monohydrate | Agar/broth supplement |
| Anaerobic sachets | Generation of anaerobic |
| | environment for jars |
| Anaerobic Det strips | Generation of anaerobic |
| | environment. |
| Catalase from Bovine liver | Agar supplementation - Used for |
| (lyophilized powder, 2000-5000 | suspension and spread plate |
| units/mg protein) | |
| Catalase Aspergillus Niger (15,500 | Agar Additive/Neutralising agent |
| units/mg protein) | |
| Sodium pyruvate | Agar and suspension/broth |
| | Supplement |
| D - (+) Trehalose dihydrate | Agar and suspension/broth |
| | Supplement |

### Weighing & Hydration of Finished Product (UHT Beverage/Food Product)

For all beverage samples, mix bottle for 1 min
For food products, dissolve the contents at room temperature/mortar+pestle to disintegrate the food structure.
Stir for 30 minutes at 250 RPM at room temperature.

### Initial 2-Stage Homogenisation and Rest Step (Homogenise Phase I).

Homogenise the bag contents for 150 sec at 480 Strokes per minute.
Allow the bag to rest at room temperature for 15 min
After rest step, repeat homogenization step

### Enzyme Digestion

After rest step, make 1 in 10 dilution with MRS broth fortified with filter sterilised additives (sodium pyruvate, D-(+) Trehalose, L-Cysteine) and catalase
Incubate for 3 h 30 minutes at 37° C. The incubation time will be dependent on product composition and viscosity. The Incubation can be anaerobic or aerobic, which is also dependent on the product composition
After 3.5 hours, add 200 μL of Protease enzyme and incubate at 37° C.
After final digestion, remove the samples from the incubator and homogenise the sample for 150 sec at room temperature.
Samples are ready for serial dilution
Serial dilutions are plated as per the expected cell dose in the finished product i.e. yogurts are anticipated to be 10⁶ CFU per mL; hence plating 10⁻², 10⁻³, 10⁻⁴.
Samples can be spread or pour plated as follows.
   Dry spread plates inoculated with catalase in laminar hood for approx 20 minutes prior to sample serial dilution.
   Alternatively ensure that agar for pour plates is tempering in water batch at 42.5° C.

### Spread Plate Preparation

Add catalase stock solution to each plate surface.
Using a hockey stick, spread catalase across surface of agar plate and avoid hockey stick hitting the walls of agar plate.
Allow the plate to dry for 10 minutes.
After catalase is dried on the plate surface, add dilution sample onto spread plates (which already contained the catalase on the plate surface).
Using a hockey stick, spread sample across surface of agar plate and avoid hockey stick hitting the walls of agar plate.
Allow the plate to dry for 10 minutes in laminar hood.

### Pour Plate Preparation

Add the sample direct onto empty Petri dish with catalase. Once the mmMRS
agar has cooled to a safe temperature to maintain enzyme activity, pour the agar into Petri dish
Swirl the plate to enable uniform distribution of the enzyme in the molten agar. This step must be conducted quickly and efficiently to prevent pre-mature solidification of agar prior to distribution of the enzyme.
Allow plates to dry for 15-20 min.

### Incubation

Transfer plates to an anaerobic jar for incubation at 37° C.
Do not invert the plates (catalase will promote the breakdown of H₂O₂ and other reactive oxygen species (ROS); hence water will form on the surface.
Anaerobic conditions will require an anaerobic jar, anaerobic sachet, and anaerobic detection strips. Adhere to manufacturer's instructions.

TABLE 3
Media required.

| | | | Incubation |
|---|---|---|---|
| Culture | Media | Reconstitution | Conditions |
| Bifidobacterium | TOS-Propionate | As per | 37° C. |
| lactis | agar | manufacturer's | Anaerobically |
| | | instructions | for 72 h |
| Lactobacillus | MRS Agar | | 37° C. |
| rhamnosus | | | Anaerobically |
| | | | for 72 h |

### Description of Controls Implemented

### Negative Controls

Inoculate agar plates with samples of sterile buffers, media additives (L-cysteine, sodium pyruvate, catalase and blank agar plates and incubate under similar conditions to the food/beverage test samples. No growth should be observed on this sample.

Test Catalase Stock Solution for Enzyme Activity Test i.e. Bubble Test or Kit Positive Controls

Inoculate one of the media plates with encapsulated Lactobacillus as the expected dose to verify the growth of the theoretical dosed amount. Incubate under similar conditions as the yogurt test samples (37° C.; 72 hours). Growth should be observed on this sample at the dosed amount.

TABLE 4
The impact of cell recovery of viable culturable cells after treatment
of heat treated samples with catalase, protease and pyruvate.
The extraction of no treatments vs extraction with protease +
catalase + sodium pyruvate treatments are compared.

| | | | Standard | |
|---|---|---|---|---|
| | | | Extraction + | |
| | | Standard | Protease + | |
| | Standard | extraction + | Catalase + | |
| Product | extraction | protease | Pyruvate | Dose |
| Fermented dairy | 0.00E+00 | 1.50E+04 | 4.45E+07 | 4.50E+07 |
| beverage | | | | |
| Fermented non- | 0.00E+00 | 2.30E+03 | 4.41E+07 | 4.50E+07 |
| dairy beverage | | | | |
| UHT dairy beverage | 0.00E+00 | 2.45E+04 | 2.39E+07 | 2.50E+07 |
| UHT non- | 0.00E+00 | 5.60E+03 | 2.45E+07 | 2.50E+07 |
| dairy beverage | | | | |
| Food | 9.10E+02 | 4.10E+05 | 1.48E+08 | 1.50E+08 |
| product (snack) | | | | |
| Food | 2.30E+03 | 8.70E+06 | 2.01E+09 | 2.00E+09 |
| product (nutritional | | | | |
| powder) | | | | |

All relevant controls were implemented data represented n=12 data replicates for each product type.

### EXAMPLES

### Example 1

### Assaying a Heat Treated Sample for Enumeration Using a Spread Plate Method.

Prepare the suspension containing the sample protease, with or without catalase and with or without sodium pyruvate and/or D-(+) Trehalose.
Perform the first homogenisation step.
Incubate for 2.5-4.5 hours at 37° C. aerobic or anaerobically (depending on the product composition)
Perform the second homogenisation step.
Perform a serial dilution in the culture medium containing/not containing sodium pyruvate.
Performing spread plate method wherein the catalase is spread on the surface and subsequently the aliquot is spread on the surface of the agar plate containing pyruvate and D-(+) Trehalose.
Incubate for 72 hours at 37° C.
Perform enumeration by counting colonies formed on the surface of the agar plate.

### Example 2

Assaying a Heat Treated Sample for Enumeration Using a Spread Plate Method with Agar Containing Sodium Pyruvate and Further Using Protease to Release the Microparticles.
Prepare the suspension containing the sample protease, with or without catalase and with or without sodium pyruvate and/or D-(+) Trehalose.
Perform the first homogenisation step.
Incubate for 2.5-4.5 hours at 37° C. aerobic or anaerobically (depending on the product composition)
Add protease to the suspension.
Incubate for 30 min at 37° C. stirring at 250 RPM.
Perform another homogenisation step.
Perform a serial dilution in the culture medium containing/not containing sodium pyruvate and/or D-(+) Trehalose.
Performing spread plate method wherein the catalase is spread on the surface and subsequently the sample is spread on the surface of the agar plate containing pyruvate and D-(+) Trehalose.
Incubate for 72 hours at 37° C.
Perform enumeration by counting colonies formed on the surface of the agar plate.

### Example 3

Assaying a Heat Treated Sample for Enumeration Using a Spread Plate Method with Agar Containing Sodium Pyruvate with the Use of an Overlay Approach.
Prepare the suspension containing the sample, protease, with or without catalase and with or without sodium pyruvate and/or D-(+) Trehalose.
Perform the homogenisation step.
Incubate for 2.5-4.5 hours at 37° C. aerobic or anaerobically (depending on the product composition)
Perform the second homogenisation step.
Perform a serial dilution in the culture medium containing/not containing sodium pyruvate and/or D-(+) Trehalose.
Performing spread plate method wherein the catalase is spread on the surface and subsequently the sample is spread on the surface of the agar plate containing pyruvate and D-(+) Trehalose, and further adding an agar overlay.
Incubate for 72 hours at 37° C.
Perform enumeration by counting colonies formed on the surface of the agar plate.

### Example 4

Assaying a Heat Treated Sample for Enumeration Using a Spread Plate Method with Agar Containing Sodium Pyruvate with the Use of an Overlay Approach. Further Using an Additional Protease Digestion Step.
Prepare a suspension containing the sample, protease, with or without catalase and with or without sodium pyruvate and/or D-(+) Trehalose.
Perform the first homogenisation step.
Incubate for 2.5-4.5 hours at 37° C., aerobic or anaerobically (depending on the product composition)
Add protease to the suspension.
Incubate for 30 min at 37° C. stirring at 250 RPM.
Perform the second homogenisation step.
Perform a serial dilution in the culture medium containing/not containing sodium pyruvate.
Performing spread plate method wherein the catalase is spread on the surface and subsequently the sample is spread on the surface of the agar plate containing pyruvate and D-(+) Trehalose and further adding an agar overlay.
Incubate for 72 hours at 37° C.
Perform enumeration by counting colonies formed on the surface of the agar plate.

### Example 5

Assaying a Heat Treated Sample for Enumeration Using a Spread Plate Method with Immobilised Catalase and Sodium Pyruvate on the Agar.
Prepare the suspension containing the sample, protease, with or without catalase and with or without sodium pyruvate and/or D-(+) Trehalose.
Perform the first homogenisation step.
Incubate for 2.5-4.5 hours at 37° C., aerobic or anaerobically (depending on the product composition)
Perform the second homogenisation step.
Perform a serial dilution in the culture medium containing/not containing sodium pyruvate and/or D-(+) Trehalose.
Performing spread plate method wherein the catalase and the sodium pyruvate is spread on the surface and subsequently the sample is spread on the surface of the plate and further adding an agar overlay.
Incubate for 72 hours at 37° C.
Perform enumeration by counting colonies formed on the surface of the agar plate.

### Example 6

Assaying a Heat Treated Sample for Enumeration Using a Spread Plate Method with Immobilised Catalase and Sodium Pyruvate on the Agar. Further with a Protease Digestion Step.
Prepare suspension containing the sample, protease, with or without catalase and with or without sodium pyruvate and/or D-(+) Trehalose.
Perform the first homogenisation step.
Incubate for 2.5-4.5 hours at 37° C., aerobic or anaerobically (depending on the product composition)
Add protease to the suspension.
Incubate for 30 minutes at 37° C. stirring at 250 RPM.
Perform the second homogenisation step.
Perform a serial dilution in the culture medium containing/not containing sodium pyruvate.
Performing spread plate method wherein the catalase and sodium pyruvate is spread on the surface and subsequently the sample is spread on the surface of the agar plate with/without a D-(+) Trehalose. Further adding an agar overlay.
Incubate for 72 hours at 37° C.
Perform enumeration by counting colonies formed on the surface of the agar plate.

### Example 7

### Assaying a Heat Treated Sample for Enumeration Using a Pour Plate Method Using Agar.

Prepare the suspension containing the sample protease, with or without catalase and with or without sodium pyruvate and/or D-(+) Trehalose.
Perform the first homogenisation step.
Incubate for 2.5-4.5 hours at 37° C., aerobic or anaerobically (depending on the product composition)
Perform the second homogenisation step.
Perform a serial dilution in the culture medium containing/not containing sodium pyruvate.
Performing pour plate method wherein the catalase and sodium pyruvate and sample are added to an empty petri dish and tempered agar with/without D-(+) Trehalose is added to the petri dish thereafter and swirled to mix.
Incubate for 72 hours at 37° C.
Perform enumeration by counting colonies formed on the surface of the agar plate.

### Example 8

Assaying a Heat Treated Sample for Enumeration Using a Pour Plate Method with a Protease Digestion Step.
Prepare the suspension containing the sample, protease, with or without catalase and with or without sodium pyruvate and/or D-(+) Trehalose.
Perform the first homogenisation step.
Incubate for 2.5-4.5 hours at 37° C. (aerobic or anaerobically (depending on the product composition)
Add the protease.
Incubate for 30 minutes at 37° C. stirring at 250 RPM.
Perform the second homogenisation step.
Perform a serial dilution in the culture medium containing/not containing sodium pyruvate and/or D-(+) Trehalose.
Performing pour plate method wherein the catalase and sodium pyruvate and sample are added to an empty petri dish and tempered agar with/without D-(+) Trehalose is added to the petri dish thereafter and swirled to mix.
Incubate for 72 hours at 37° C.
Perform enumeration by counting colonies formed on the surface of the agar plate.

### Example 9

Assaying a Heat Treated Sample for Enumeration Using a Pour Plate Method with an Agar Containing Catalase and Sodium Pyruvate.
Prepare the suspension containing the sample, protease, with or without catalase and with or without sodium pyruvate and/or D-(+) Trehalose.
Perform the first homogenisation step.
Incubate for 3.5 hours at 37° C. aerobic or anaerobically (depending on the product composition)
Perform the second homogenisation step.
Perform a serial dilution in the culture medium containing/not containing sodium pyruvate.
Performing pour plate method the sample is added to the empty petri dish and tempered agar with/without D-(+) Trehalose containing catalase and pyruvate is added to the petri dish thereafter and swirled to mix.
Incubate for 72 hours at 37° C.
Perform enumeration by counting colonies formed on the surface of the agar plate.

### Example 10

Assaying a Heat Treated Sample for Enumeration Using a Pour Plate Method with an Agar Containing Catalase and Sodium Pyruvate with an Additional Protease Digestion Step.
Prepare the suspension containing protease, with or without catalase and with or without sodium pyruvate and/or D-(+) Trehalose.
Perform the first homogenisation step.
Incubate for 2.5-4.5 hours at 37° C., aerobic or anaerobically (depending on the product composition)
Add the protease.
Incubate for 30 minutes at 37° C.
Perform the second homogenisation step.
Perform a serial dilution in the culture medium containing/not containing sodium pyruvate.
Performing pour plate method the sample is added to the empty petri dish and tempered agar with/without D-(+) Trehalose containing catalase and pyruvate is added to the petri dish thereafter and swirled to mix.
Incubate for 72 hours at 37° C.
Perform enumeration by counting colonies formed on the surface of the agar plate.

### Summary of Catalase and Pyruvate Interactions

Catalase and sodium pyruvate can work together in cellular metabolism, but their functions and interactions are part of complex biochemical pathways as follows:

### Catalase Function

Catalase is an enzyme found in nearly all living organisms exposed to oxygen. It catalyses the decomposition of hydrogen peroxide to water and oxygen. Hydrogen peroxide is a byproduct of many cellular reactions and can be harmful to cells if not removed or reduced because it can lead to oxidative damage.

Catalase is an enzyme that catalyses the decomposition of hydrogen peroxide into water and oxygen. When inoculated into MRS (de Man, Rogosa, and Sharpe) broth, which is designed to support the growth of lactic acid bacteria, catalase could degrade or lose activity over time due to several factors:

### Temperature Sensitivity

Catalase has an optimal temperature range for its activity. Incubation at 37° C. is generally within the optimal range for many catalase enzymes, but prolonged exposure at this temperature can lead to gradual denaturation and loss of activity. Hence it is important to maintain broth and agar temperatures as per the required protocol.

### pH Changes

MRS broth is slightly acidic, which is suitable for the growth of lactic acid bacteria. However, if the pH drops further due to bacterial metabolism (as lactic acid is produced), it can lead to the denaturation of catalase, as the enzyme is sensitive to pH changes. Hence the product composition is important to consider for the incubation steps and where it will be anaerobic or aerobic.

### Proteolytic Enzymes

Some bacteria produce proteases that can degrade proteins, including enzymes like catalase. If proteolytic bacteria are present in the MRS broth, they might produce enzymes that degrade catalase over time. Hence the product composition will need to be considered and this will influence the decision to incubate aerobic or anaerobically.

### Presence of Reducing Agents

Certain reducing agents or compounds in the broth reduce the disulfide bonds in catalase, leading to loss of its tertiary structure and subsequent degradation. Hence the product composition and type of broth utilised is a key factor for the optimal incubation. These factors can contribute to the degradation or inactivation of catalase when it is inoculated in nutrient broth and incubated at 37° C. Hence, product composition is assessed to determine if anaerobic or aerobic incubation is optimal for analysis.

Catalase is added to nutrient broth and agar several purposes:

### Counteract Hydrogen Peroxide

Lactic acid bacteria and starter cultures produce hydrogen peroxide, especially under stress conditions, which can inhibit their own growth and other probiotics in the same product environment. Adding catalase to analysis test, helps to degrade hydrogen peroxide, thus preventing its inhibitory effects on enumeration of probiotic in the sample product.

### Assess Microbial Response

Catalase is used to study the response of probiotic strains to oxidative stress and stress response of bacteria with and without encapsulation in finished products. Furthermore, it can assess microbial robustness independent of the effects of hydrogen peroxide in the environment.

### Enhance Growth of Sensitive Strains

Most probiotics are sensitive to oxidative stress, especially after production process related to finished food products. Addition of catalase to the assay test, can improve the growth of these sensitive strains by breaking down hydrogen peroxide that might otherwise accumulate and inhibit their growth during the enumeration assay. Hence the addition of catalase endorses a better assay for true quantification of viable cultures in a finished food product.

Sodium pyruvate is a salt form of pyruvate, which is a key intermediate in several metabolic pathways, including glycolysis. Pyruvate plays a pivotal role in energy production processes within the cell. It can be converted into acetyl-CoA and enter the citric acid cycle (Krebs cycle) for further energy production, or it can be used in the process of gluconeogenesis to produce glucose.

### Interaction of Catalase and Sodium Pyruvate

While catalase and sodium pyruvate are involved in different parts of cellular metabolism, they can be linked through the cellular response to oxidative stress. Pyruvate can act as an antioxidant, scavenging hydrogen peroxide and converting it into water and carbon dioxide, which complements the action of catalase in protecting the cell from oxidative damage. Furthermore, the generation of hydrogen peroxide and its subsequent detoxification by catalase can indirectly affect the metabolic pathways in which pyruvate is involved by influencing the cellular redox state.

The direct “working together” of catalase and sodium pyruvate is more about their complementary roles in maintaining cellular health and less about a direct interaction between the two molecules. Hence, they are synergistic but work in separate, independent pathways. They both play roles in the cell response to oxidative stress, although they are involved in different pathways and mechanisms. Hence this invention presents a novel approach to combine the effects of catalase and sodium pyruvate for the recovery of viable culturable cells from processed food and beverages fortified with encapsulated probiotics.

Adding pyruvate to agar can promote cell growth by acting as an additional carbon source and mitigating oxidation stress. Addition as a suspension acts to mitigate oxygen stress also. Addition to our suspension would enable direct cell uptake and utilisation during the incubation pre plating and also act as an energy source.

Hence the following invention involved the addition of both pyruvate and catalase to the sample suspension and agar plate to promote the resuscitation of cells from cultivation from finished product samples.

### EQUIVALENTS

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. A method of assaying a heat treated sample containing probiotic bacteria to enumerate viable culturable probiotic bacteria in the heat treated sample, comprising:
providing a suspension comprising the heat treated sample and nutrient broth;
adding an aliquot of the suspension and agar to a culture plate by a pour plate or spread plate method to provide a culture medium;
incubating the culture medium to allow probiotic bacterial colonies to grow in the culture medium; and
enumerating the probiotic bacterial colonies,
wherein the culture medium comprises added catalase and a pyruvate salt.

2. The method according to claim 1, including the step of incubating the suspension prior to adding an aliquot of the incubated suspension and agar to a culture plate.

3. The method according to claim 2, in which the probiotic bacteria in the heat treated sample are provided in the form of microparticles comprising probiotic bacteria encapsulated in a protein matrix, and in which the suspension comprises a protease enzyme configured to release probiotic bacteria from the microparticles.

4. The method according to claim 1, wherein the concentration of added catalase in the culture medium is 100-250 U per millilitre culture medium.

5. The method according to claim 1, wherein the concentration of the pyruvate salt in the culture medium is 10-30 mM.

6. The method according to claim 1, wherein the catalase and pyruvate salt is added to the suspension and/or the agar.

7. The method according to claim 2, wherein the suspension is homogenised before and/or after the first incubation step.

8. The method according to claim 3, wherein the protease is combined with the heat treated sample, nutrient broth and added catalase and pyruvate salt during a first incubation step.

9. The method according to claim 1, wherein the aliquot of the incubated suspension is prepared by serial dilution.

10. The method according to claim 1, wherein the culture medium comprises added L-cysteine.

11. The method according to claim 1, wherein the heat treated sample is a UHT beverage.

12. The method according to claim 1, wherein the probiotic bacteria are selected from Bifidobacteria and Lactobacillus.

13. The method according to claim 1, wherein the added catalase is added to the agar at 40 to 45° C.

14. The method according to claim 1, in which the culture plate is prepared by the spread plate method, in which the method comprises the steps of pouring a first layer of agar, allowing the first agar layer to solidify, spreading the aliquot of the optionally incubated suspension on a top of the first agar layer, and pouring a second agar layer on top of the first agar layer.

15. The method according to claim 1, in which:
the catalase and pyruvate salt are added to the suspension and the agar;
the concentration of added catalase in the culture medium is 100-250 U per millilitre culture medium; and
the concentration of the pyruvate salt in the culture medium is 10-30 mM.

16. A culture medium comprising agar, nutrient broth, added catalase, added pyruvate salt, and an inoculum comprising probiotic bacteria.

17. The culture medium according to claim 16, in which the culture medium comprises 100-250 U catalase per millilitre of culture medium and 10-30 mM pyruvate salt.

18. The culture medium according to claim 16, in which the inoculum comprises a hydrolysate obtained by digesting microparticles comprising probiotic bacteria encapsulated in a protein matrix with a protease.

19. The culture medium according to claim 16, in which the culture medium comprises added L-cysteine.

20. A culture plate comprising a culture medium of claim 16.

21. The culture plate according to claim 20, prepared by a pour plate or spread plate method.
